(19) United States
(12) Patent Application Publication
(10) Pub. No.: US 2024/0165304 A1
(43) Pub. Date: May 23, 2024

(54) COMPOSITE WIRE WITH POWDER CORE

(71) Applicant: FORT WAYNE METALS RESEARCH PRODUCTS, LLC, Fort Wayne, IN (US)

(72) Inventors: Adam J. Griebel, Fort Wayne, IN (US); Jeremy E. Schaffer, Fort Wayne, IN (US)

(21) Appl. No.: 18/284,375

(22) PCT Filed: Mar. 29, 2022
(86) PCT No.: PCT/US2022/022343

§ 371 Date: Sep. 27, 2023

Related U.S. Application Data
(60) Provisional application No. 63/167,942, filed on Mar. 30, 2021.

(51) Int. Cl. A61L 31/02; A61L 31/18

(52) U.S. Cl. CPC A61L 31/022; A61L 31/18

(57) ABSTRACT

A composite wire uses a powder core to offer radiopaque enhancements without the drawbacks of a solid metal core. For example, composite wires may include highly radiopaque materials, such as tantalum and platinum, integrated into the wire core in powder form. The powder core provides high radiopacity to the finished wire while preserving mechanical properties of the shell material. The powder form of the core material also enables a wider range of candidate materials for the composite wire core, such that a desirable electrochemical profile may be maintained between the core and shell materials, including bioabsorbable shell materials.

### CROSS-REFERENCE TO RELATED APPLICATION

This application claims the benefit of U.S. Provisional Application Ser. No. 63/167,942, filed Mar. 30, 2021 and entitled COMPOSITE WIRE WITH ENHANCED RADIOPACITY, ELASTICITY, AND ELECTROCHEMICAL BEHAVIOR, the entire disclosure of which is hereby incorporated herein by reference.

### BACKGROUND

### 1. Technical Field

The present disclosure relates to composite wires and, in particular, to composite wires with a solid shell and a powder core.

### 2. Description of the Related Art

Minimally invasive surgical procedures in which devices are delivered to the target site via guidewires and catheters often rely on real-time imaging methods such as fluoroscopy to enable accurate placement and ensure proper deployment. To be distinguished from fluoroscopic image background, devices must have a certain amount of radiopacity, or ability to block and scatter x-rays. Radiopacity is thus an important property and can be challenging to achieve in thin material sections.

Radiopacity is largely a function of three things: section thickness, material density, and material atomic number. For small devices like stents with very thin struts (e.g. 0.3 to 0.02 mm), proper material selection is required to account for lack of section thickness. Table 1, material selection can have a large impact on device radiopacity. Platinum, for example, has nearly 5 times the density of titanium, and 12 times the density of magnesium. Nitinol (i.e., a roughly equiatomic nickel-titanium alloy) also has relatively low density compared to stainless steel, cobalt chrome, tantalum, or platinum.

TABLE 1
Material properties of medically relevant metals

| | Density | Major Element |
|---|---|---|
| Alloy | (g/cc) | Atomic Number |
| Mg | 1.74 | 12 |
| CP Titanium | 4.5 | 22 |
| Nitinol | 6.45 | 28/22 |
| Stainless Steel | 7.87 | 26 |
| Cobalt Chrome | 9.13 | 27 |
| Tantalum | 16.65 | 73 |
| Gold | 19.3 | 79 |
| Platinum | 21.45 | 78 |

Several strategies have been employed to achieve adequate combinations of radiopacity, mechanical strength, biocompatibility, and cost. Some devices achieve sufficient radiopacity without specific enhancements. Other devices will incorporate radiopaque (e.g. Pt, Ta, Au) markers as secondary add-ons, or as a portion of the wires of a multi-wire construct. In some cases, alloy compositions can be tuned to enhance radiopacity with additions of radiopaque elements. An increasingly common strategy to improve radiopacity of an entire device without the need to modify alloy compositions or add on secondary markers is to use a DFT™ composite wire produced by Fort Wayne Metals.

Composite wires with a core of one material and a shell of another material can be used to produce wires which advantageously combine the properties of two or more materials. One such composite wire is DFT® wire available from Fort Wayne Metals Research Products, LLC of Fort Wayne, Indiana. Generally speaking, composite wires may be produced and used in sizes ranging from 0.01 mm up to 1 mm. Further details regarding the production of composite and DFT® constructs, and resulting wire shapes and sizes, are discussed in U.S. Pat. No. 7,989,703, filed Feb. 27, 2009, entitled “Alternating Core Composite Wire”, and in U.S. Pat. No. 9,561,308 filed on Jun. 24, 2011 and entitled “Biodegradable Composite Wire For Medical Devices,” the entire disclosures of which are incorporated by reference herein.

Wire radiopacity is an important aspect of stent design and can be enhanced through DFT® wire compositing. However, this approach can have limitations, such as mechanical performance limitations in nitinol wire and electrochemical limitations in magnesium wire.

What is needed is an improvement over the foregoing.

### SUMMARY

A composite wire uses a powder core to offer radiopaque enhancements without the drawbacks of a solid metal core. For example, composite wires may include highly radiopaque materials, such as tantalum and platinum, integrated into the wire core in powder form. The powder core provides high radiopacity to the finished wire while preserving mechanical properties of the shell material. The powder form of the core material also enables a wider range of candidate materials for the composite wire core, such that a desirable electrochemical profile may be maintained between the core and shell materials, including bioabsorbable shell materials.

In one form thereof, the present disclosure provides a composite wire including a shell made of a solid metallic material, the shell defining an outer diameter and a hollow cavity extending longitudinally along the shell and defining an inner diameter, and a core made of a non-metallic powder material received within the hollow cavity and defining an outer diameter equal to the inner diameter.

In another form thereof, the present disclosure provides a drawn-filled tube wire construct, including a shell made of a shell material, the shell defining an outer diameter and a hollow cavity extending longitudinally along the shell and defining an inner diameter, and a core of made of a powder material received within the hollow cavity and defining an outer diameter equal to the inner diameter, wherein the non-metallic powder of the core has a higher radiopacity than the metallic material of the shell such that the wire has a radiopacity at least 100% greater than an equivalent solid wire made of the shell material only.

In yet another form thereof, the present disclosure provides a drawn-filled tube wire construct, including a shell made of a shell material, the shell defining an outer diameter and a hollow cavity extending longitudinally along the shell and defining an inner diameter, and a core of made of a powder material received within the hollow cavity and defining an outer diameter equal to the inner diameter, the powder material forming a galvanic couple with the shell material defining a voltage of less than 0.5V.

In still another form thereof, the present disclosure provides a wire for a medical device including a shell made of a shell material, the shell defining an outer diameter and a hollow cavity extending longitudinally along the shell and defining an inner diameter, the shell made of an absorbable metallic material, and a core received within the hollow cavity and defining an outer diameter equal to the inner diameter the core made of a non-metallic powder. The non-metallic powder of the core has a higher radiopacity than the metallic material of the shell such that the wire has a radiopacity at least 100% greater than an equivalent solid wire made of the shell material only. The core and the shell have a galvanic couple of less than 0.1V. The wire is configured to transition in vivo from a non-degraded configuration in which the shell is in solid form and encapsulates the non-metallic powder of the core, to a fully-degraded configuration in which the shell is absorbed and the non-metallic powder is exposed, the wire having a first mechanical strength in the non-degraded configuration and a substantially zero mechanical strength in the fully-degraded configuration.

In yet another form thereof, the present disclosure provides a wire for a medical device including a shell made of a shell material, the shell defining an outer diameter and a hollow cavity extending longitudinally along the shell and defining an inner diameter, the shell made of a superelastic metallic material, and a core received within the hollow cavity and defining an outer diameter equal to the inner diameter the core made of a non-metallic powder. The non-metallic powder of the core has a higher radiopacity than the metallic material of the shell such that the wire has a radiopacity at least 100% greater than an equivalent solid wire made of the shell material only. The core and the shell have a galvanic couple of less than 0.1V. The wire defines a first isothermally recoverable strain which is substantially identical to a second isothermally recoverable strain of the equivalent solid wire made of the shell material only, whereby the core provides no mechanical resistance to a superelastic function of the shell such that the shell recovers from a mechanical deformation to the same extent as an equivalent diameter solid wire of the superelastic metallic material would recover from the mechanical deformation.

### BRIEF DESCRIPTION OF THE DRAWINGS

The above mentioned and other features of this invention, and the manner of attaining them, will become more apparent and the invention itself will be better understood by reference to the following description of embodiments of the invention taken in conjunction with the accompanying drawings, where:

FIG. 1 is a schematic cross-section of a composite wire made in accordance with the present disclosure, including a radiopaque powder core;

FIG. 2 is an X-ray photograph of a series of wires including three samples of predicate materials and one sample, shown at right, of a material in accordance with the present disclosure;

FIG. 3 is an X-ray photograph of a pair coiled wires including one sample of predicate material at the top, and one sample of a material in accordance with the present disclosure shown at the bottom;

FIG. 4 is a photograph of a cross-section of a composite wire made in accordance with the present disclosure;

FIG. 5 is an X-ray photograph of a series of wires including three samples of material in accordance with the present disclosure and one sample, shown at top, of a predicate material;

FIG. 6 is an X-ray photograph of a wire in accordance with the present disclosure wrapped around a construct made of predicate wires;

FIG. 7 is a cross-sectional view of a pair of wires made in accordance with the present disclosure;

FIG. 8 is a stress-strain graph showing performance characteristics of a wire made in accordance with the present disclosure, juxtaposed with performance characteristics of a predicate wire;

FIG. 9 is a stress-strain graph showing performance characteristics of a pair of wires made in accordance with the present disclosure, juxtaposed with performance characteristics of a predicate wire;

FIG. 10 is an X-ray photograph of a series of wires including one sample, shown at bottom, of material in accordance with the present disclosure and three samples of a predicate material;

FIG. 11 is a photograph of a cross-section of three composite wires made in accordance with the present disclosure;

FIG. 12 is an X-ray photograph of a series of coiled wires including three samples of material in accordance with the present disclosure and two samples of a predicate material;

FIG. 13 is an X-ray photograph of a series of wires including three samples of material in accordance with the present disclosure and two samples of a predicate material;

FIG. 14 is an X-ray photograph of a series of wires including one sample of material in accordance with the present disclosure and two samples of a predicate material, with each of the three total samples being subjected to two different X-rays;

FIG. 15 is a schematic view illustrating an exemplary process of forming composite wire using a lubricated drawing die;

FIG. 16 is an elevation view of a wire in accordance with the present disclosure, before a final cold working process; and

FIG. 17 is an elevation view of the wire of FIG. 16, after the final cold working process.

Corresponding reference characters indicate corresponding parts throughout the several views. Unless stated otherwise the drawings are to scale and proportional.

### DETAILED DESCRIPTION

The embodiments disclosed below are not intended to be exhaustive or to limit the invention to the precise forms disclosed in the following detailed description. Rather, the embodiments are chosen and described so that others skilled in the art may utilize their teachings.

As used herein, “wire” or “wire product” encompasses continuous wire and wire products which may be continuously produced and wound onto a spool for later dispensation and use, such as wire having a round cross section and wire having a non-round cross section, including flat wire or ribbon. “Wire” or “wire product” also encompasses other wire-based products such as strands, cables, coil, and tubing, which may be produced at a particular length depending on a particular application. Although round cross-sectional wire forms are shown in the Figures of the present application and described further below, non-round wire forms may also be produced in accordance with the present disclosure. Exemplary non-round forms include polygonal cross-sectional shapes such as rectangular cross-sectional shapes.

“Fine wire” refers to a wire having an outer diameter of less than 1 mm. “Ultrafine wire” refers to a wire having an outer diameter of 50 μm or less.

“Superelastic” material is material which is capable of undergoing strain exceeding 2% with negligible plastic deformation, such that the material is able to return to its original dimension after the deformation without permanent damage.

“Isothermally recoverable strain” is recoverable strain observable in a substantially constant ambient temperature, i.e., without external heating or cooling of the work piece. The work piece may experience some internal heating or cooling from microstructural changes within an isothermal strain recovery. Ambient temperature may vary by a small amount during isothermal strain recovery, such as plus-or-minus 3° C. from the nominal temperature at the start of the stain recovery. Ambient temperature may be room temperature, i.e., 20-30° C., or body temperature, i.e., 36.4-37.2° C.

“DFT®” is a registered trademark of Fort Wayne Metals Research Products Corp. of Fort Wayne, IN, and refers to a bimetal or poly-metal composite wire product including two or more concentric layers of materials, typically at least one outer layer or shell disposed over a core filament, and formed by drawing a tube or multiple tube layers over a core element.

“Impurities,” “incidental impurities” and “trace impurities” are material constituents present in a material at less than 500 parts per million or 0.05 wt. %. Alloys “free” of or “excluding” a certain constituent are alloys having such a constituent in amounts equal to or less the 500 parts per million impurities threshold.

1. Composite Wire with Powder Core

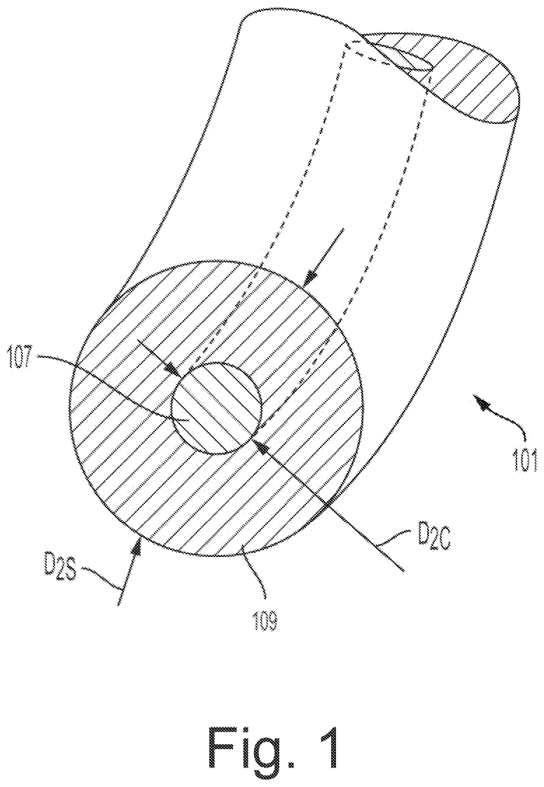

FIG. 1 shows a composite wire 101 having a core 107 and a shell 109 surrounding the core 107. In an exemplary embodiment, core 107 and shell 109 each define longitudinal axes which are coaxial, i.e., the core 107 is centered in the shell 109. More particularly, the shell 109 defines a hollow cavity extending longitudinally along its length, and core 107 is received within and completely fills the hollow cavity, such that an inner diameter of defined by the cavity and the outer diameter of the core 107 are the same, i.e., diameter D_{2C} as shown in FIG. 1

Core 107 is made of a powder material as further described below, with the powder consisting of fine, dry grains or particles of a selected material or combination of materials, in which the grains are not chemically bonded to one another such that the powder can flow as a non-Newtonian fluid. In one embodiment, the grains of the powder core 107 have a substantially consistent size relative to one another, such as a maximum size variation of 10% across the grains of the core 107. The grains of the powder core 107 fully and completely occupy the hollow core formed within shell 109 with a substantially consistent density of grains along the entire length of wire 101. In an exemplary embodiment, the density may vary by no more than 1%, 5% or 10% across the entire volume of core 107. In a further exemplary embodiment, the grains of the powder core 107 may have a mean particle size less than 10 microns, less than 5 microns, or about 1 micron.

As also described below, the powdered form of core 107 allows the core 107 to be “flexible” insofar as the grains of the powder can freely rearrange relative to one another in a way that the material of a solid-metal core cannot. This allows core 107 to minimally affect the mechanical performance of wire 101, such as strength, ductility and fatigue resistance, such that the mechanical properties of the wire 101 may be defined by shell 109 alone. Stated another way, powder core 107 made in accordance with the present disclosure may be considered to be “mechanically invisible” in the context of wire 101 where wire 101 including powder core 107 and shell 109 experiences a minimal change in ductility, strength, fatigue endurance, plateau stress levels and/or permanent set (also referred to herein as residual strain) as compared to a reference of the same material and configuration of shell 109 lacking core 107 (i.e., with a hollow core). For purposes of the present disclosure, such a minimal change may be a change of 5%, 3%, 1% or less.

Composite wire 101 may be used in medical devices designed for use in the human body. If so, core 107 and shell 109 are formed exclusively of medical-grade materials, which are materials approved or otherwise suitable for use in implantable or in vivo medical devices. “Medical-grade” materials specifically exclude certain materials not suitable for use in, or in connection with medical procedures on, the human body. Examples of non-medical grade materials are materials not suitable for contact with tissue and/or blood, including materials which cannot pass cytotoxicity testing of at least one hour of such contact. Non-medical grade materials include heavy metals including lead and cadmium, materials such as beryllium and beryllium copper, and any other materials generally regarded as toxic to the human body or otherwise damaging to human tissue. All materials discussed herein with respect to their potential use for shell 109 or core 107 are medical-grade materials.

For example, a composite wire using 35N LT® for the shell and Ag for the core (which may be referred to as “35N LT®-DFT-Ag” wire) is commonly used for biostimulation leads. In such an application, the cobalt-chrome 35N LT® alloy shell provides strength, fatigue resistance, and corrosion resistance, while the Ag core provides conductivity. An increasingly used construction is NiTi-DFT-Pt, in which the superelasticity of NiTi (nitinol) enables minimally invasive implantation techniques and medical devices which couldn't be delivered with other materials. As can be seen in Table 1 above, nitinol also has relatively low radiopacity. The addition of the Pt core provides radiopacity with minimal impact on mechanical performance provided the core is a sufficiently small portion of the overall wire cross section, or “core ratio”. Further discussions of core ratios can be found in U.S. Pat. No. 9,561,308, the entire disclosure of which is hereby incorporated by herein by reference. At high core ratios, a solid-wire Pt core can increasingly impede the superelastic performance of the nitinol shell due to plastic deformation resisting full recovery. Radiopacity is quantified using the pixel intensity difference method as describe in ASTM F640, the entire disclosure of which is hereby incorporated herein by reference.

In wire 101 using powder core 107, higher core ratios can be utilized while retaining the full superelastic performance of shell 109. These higher core ratios, relative to solid-core composite wires of comparable radiopacity, preserve the superelastic performance or other mechanical performance characteristics of wire 101, and can also reduce costs by replacing the core and a portion of the shell of a conventional composite wire with a lower-cost material for core 107 as described herein. For example, wires 101 may have core ratios of at least 10%, 15%, 20% or 25%, and as much as 40%, 50% or 60% within the scope of the present disclosure.

In particular, the powder core 107 may exert no influence on the mechanical performance of wire 101. In this way, wire 101 may be designed with mechanical performance characteristics that are identical or substantially identical to a solid wire made of the same superelastic material as shell 109 with the same outer diameter D_{2S} (FIG. 1). For purposes of this comparison between wire 101 and the equivalent solid wire of same material and diameter, “substantially identical” may mean a difference of less than 5%, 3% or 1% in a given performance metric. One such performance metric is isothermally recoverable strain, noted above as recoverable strain observable in a substantially constant ambient temperature. For example, wire 101 may be strained to a given level of 3%, 4%, 5%, 6% or more, for example, such that the wire 101 is “stretched” and elongated by the amount of the strain. The force on the wire 101 is then removed and wire 101 is allowed to elastically recover. The residual strain is calculated by the percentage of the permanent elongation of the wire 101 after the recovery. The maximum strain of any given configuration of wire 101 which leads to zero residual strain is the maximum isothermally recoverable strain. This metric of mechanical performance may be identical or substantially identical to the same metric applied to a solid, monolithic reference wire having the same diameter D_{2S} and being made of the same metallic material as shell 109. In this way, the core 107 provides no mechanical resistance to a superelastic function of the shell 109 such that the shell 109 recovers from a mechanical deformation to the same extent as an equivalent diameter solid wire made the same superelastic metallic material, given the same mechanical deformation. This relationship may also hold true for strains which do lead to permanent deformation, i.e., the residual strain of wire 101 may be identical or substantially identical to the residual strain in an equivalent solid wire after a given strain percentage.

Core 107 may be designed for its radiopacity relative to shell 109. In medical devices, the materials for shell 109 may be selected for mechanical performance, bioabsorbability, or other desired characteristics, while core 107 may be selected to impart radiopacity of the overall wire 101. Radiopacity may be imparted by a combination of core diameter, core material and X-ray emitting conditions, for example. In an exemplary embodiment, wire 101 made from shell 109 and core 107 has a radiopacity that is at least 20%, 40% or 100% greater than a radiopacity of an equivalent solid, monolithic wire having the same diameter and made solely from the material of shell 109. In some cases, such as for the LZ21-DFT-25% BaSO₄ wire configurations discussed in further detail below, radiopacity is between 700% and 2,000% greater than an equivalent solid wire made from the LZ21 material alone.

The use of powder cores 107 for composite wire 101 also allows materials to be used in core 107 which might otherwise be ineligible due to their effect on the mechanical performance for the overall wire 101. Stated another way, the use of powder core 107 results in an expansion of material options for the core 107 of wire 101 at a given level of mechanical performance. These new options for core 107 can be leveraged to provide a core 107 with a low electromechanical potential relative to the shell 109, thereby minimizing or eliminating galvanic interaction at the interface of core 107 and shell 109.

For example, the material in Table 1 with the lowest density is magnesium, a nutrient metal which can be used in absorbable medical devices. As can be deduced from Table 1, the radiopacity of Mg is very poor, rendering Mg stent struts invisible in most x-rays. While incorporation of a radiopaque metal core (e.g. Mg-DFT-Pt, Mg-DFT-Fe) is technically feasible, the resulting galvanic coupling between the Mg and the more noble metal core may negatively affect the dissolution profile for the Mg shell, particularly for certain in vivo applications where slow and/or controlled degradation is desired.

Additional discussion of Mg and other biodegradable wire materials useable in connection with the present DFT materials may be found in U.S. Pat. No. 9,561,308, incorporated above, and in U.S. Patent Application No. 20160138148, filed Dec. 3, 2015 and entitled BIODEGRADABLE WIRE FOR MEDICAL DEVICES, the entire disclosure of which is incorporated by reference herein. Further additional discussion of bioabsorbable materials useable in connection with the present DFT materials may be found in Absorbable Wire Radiopacity Influence of Composition and Size on Xray Visibility, by Griebel et al., the entire disclosure of which is incorporated herein by reference.

For wires 101 which include bioabsorbable shells 109, the wire 101 (or a medical device made from one or more wires 101, such as a stent) can be expected to transition from a non-degraded state to a fully-degraded state in vivo as the bioabsorbable material of shell 109 is absorbed by the body. In the non-degraded state, i.e., upon manufacture and before or immediately upon implantation in a body, shell 109 is in solid form with the powder material of core 107 fully encapsulated and unexposed to the ambient environment. In this initial, non-degraded configuration, wire 101 has a mechanical strength and other mechanical characteristics arising from its material, manufacture and configuration. For example, where wires 101 are incorporated into a stent, the strength may be suitable for radial support of a vessel wall.

Over time, a shell 109 made of a bioabsorbable material will degrade and be absorbed by the body, reducing the strength of the wire or wires 101 until, ultimately, the shell 109 is substantially or completely absorbed. At this point, the powder core 107 becomes exposed to the ambient environment around the wire 101, and may be allowed to disperse harmlessly (e.g., into the bloodstream or via phagocytosis) or remain harmlessly at the site of the implant. At this fully-degraded state, the wire 101 has substantially zero mechanical strength, as the shell 109 is either completely dissipated or merely a hollow, sinuous frame with no ability to mechanically influence the vessel wall or other structure surrounding it.

In wire 101 using powder core 107, radiopaque materials can be used for core 107 that may otherwise be ineligible in solid form for reasons of mechanical penalties, manufacturability, or other practical considerations. These core materials, discussed in additional detail below, may be particularly beneficial for a lower galvanic potential with an absorbable shell 109, though it is also contemplated that wires 101 with non-absorbable shell materials 109, such as NiTi, may also benefit from the ability to select powder cores 107 with low galvanic potential.

For wire 101, the galvanic couple between core 107 and shell 109 is negligible. In an exemplary embodiment, the inherent voltage potential between the material of core 107 and the material of shell 109 is kept to less than 0.5V, 0.3V or 0.1V. Where shell 109 is an absorbable material such as Mg or a Mg alloy, as further described below, the galvanic couple between core 107 and shell 109 may be kept to less than 0.5V. Where core 107 is an oxide or sulfate fill as described herein, the galvanic potential is 0V.

### 2. Exemplary Materials and Constructs

Powder cores 107 may be non-metallic, and these non-metallic cores 107 may be mated to metallic shells 109. Non-metallic materials include ceramics and polymers, including tungsten carbide and other compounds of metallic elements and oxygen, carbon, nitrogen, or sulfur, as well as salts. All exemplary materials discussed herein for core 107 are non-metallic.

By contrast, shell 109 may be made of a metallic material, which is any material made of metal or metal alloy and not classified as a ceramic or polymer material. All of the exemplary materials discussed herein for shell 109 are metallic.

Exemplary powders for use in core 107 of wire 101 include bismuth trioxide, tantalum oxide, and tungsten carbide, which provide very high radiopacity while also being generally recognized as safe for use in medical devices in vivo. Other powders which may be used as core 107 in a wire 101 in accordance with the present disclosure include barium sulfate, zirconium oxide, hafnium oxide, zirconium oxide, titanium oxide, niobium oxide, strontium oxide, molybdenum oxide, tungsten oxide, zinc oxide, rare earth oxides (e.g. erbium oxide, holmium oxide, gadolinium oxide, dysprosium oxide, ytterbium oxide, and neodymium oxide), iodine or iodine-based compounds (e.g. potassium iodide), bromine or bromine-based compounds (e.g. sodium bromide, potassium bromide), and the like. Mixes of radiopaque powders may also be used in accordance with the present disclosure, as required or desired for a particular application. Any of the foregoing exemplary powder materials may be mixed with any number of the other exemplary powder materials.

Powder core 107 is suitable for use with shell 109 of nearly all wire alloy classes discussed above. Exemplary shells 109 may include magnesium, zinc, iron, titanium, titanium-beta, nitinol, stainless steel, cobalt-chrome, nickel, tantalum, platinum, tungsten, and alloys thereof. Certain exemplary shell materials are described in Absorbable Wire Radiopacity: Influence of Composition and Size on X-ray Visibility by Adam J. Griebel et al., the entire disclosure of which is hereby expressly incorporated herein by reference. Selected materials are shown in Table 2 below along with their trade names, including LZ21, ZX10, and AZ31.

TABLE 2
Selected Shell Materials

| | | Density |
|---|---|---|
| Alloy | Nominal composition (wt %) | (g/cc) |
| LZ21 | Mg—2Li—1Zn | 1.68 |
| ZX10 | Mg—1Zn—0.3Ca | 1.74 |
| AZ31 | Mg—3Al—1Zn—0.2Mn | 1.77 |
| WE43 | Mg—4Y—3Nd—0.4Zr | 1.81 |
| Resoloy | Mg—10Dy—1Nd—1Zn—0.2Zr | 1.91 |
| Zn7Ag | Zn—7Ag | 7.29 |
| Fe35Mn | Fe—35Mn | 7.69 |

Additional materials suitable for shell 109 of wire 101 are discussed, for example, in U.S. Patent Application No. 2019/0292640 filed Nov. 14, 2016 and entitled NI-FREE BETA TI ALLOYS WITH SHAPE MEMORY AND SUPER-ELASTIC PROPERTIES, the entire disclosure of which is incorporated herein by reference. Additional materials useable for shell 109 of composite wire 101 in accordance with the present disclosure are discussed in Engineering Characteristics of Drawn Filled Nitinol Tube, by Schaffer et al., the entire disclosure of which is incorporated by reference herein in its entirety. Still further materials useable for shell 109 of composite wire 101 in accordance with the present disclosure are discussed in Effect of Ta on Microstructures and Mechanical Properties, by Cai et al., the entire disclosure of which is incorporated by reference herein in its entirety.

Turning again to the use of absorbable materials such as Mg for shell 109 of wire 101, a tube made of the Mg alloy identified as LZ21 in Table 2 may be filled with barium sulfate powder and drawn via conventional cold wire drawing techniques, as further described in detail below. When the drawing process is complete, composite wire 101 is produced as LZ21-DFT-25% BaSO₄, where 25% denotes 25 percent of the cross-sectional area is the powder core 107 made of barium sulfate (i.e., BaSO₄), while the remaining 75% of the cross-sectional area is the shell 109 made of LZ21.

For purposes of illustrating the radiopacity advantages offered by the LZ21-DFT-25% BaSO₄ wires, samples ranging from 0.2 to 1.0 mm were produced by various wire drawing schedules and imaged via a conventional x-ray system.

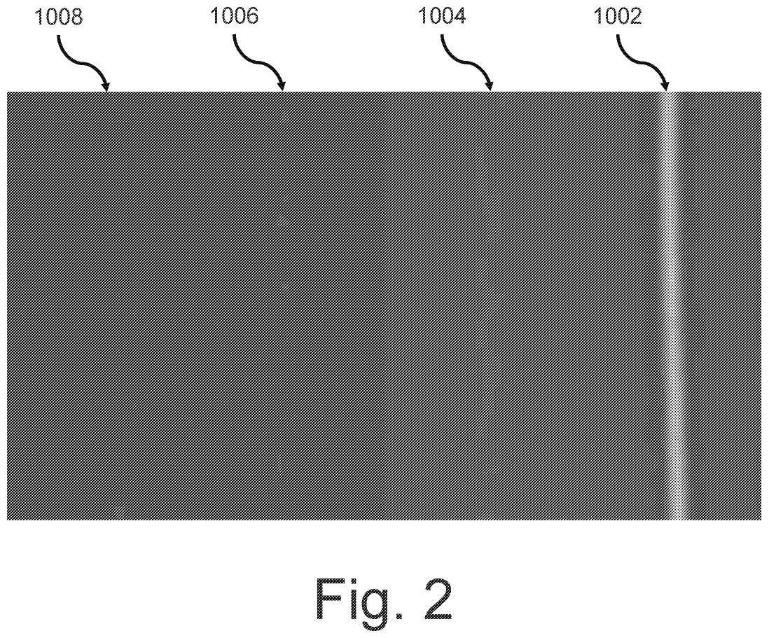

Turning to FIG. 2, four of the 1 mm wire samples are shown. Wire 1008 is a solid, monolithic wire made from ZX10. Wire 1006 is a solid, monolithic wire made from LZ21. Wire 1004 is a solid, monolithic wire made from AZ31. Wire 1002 is a composite wire in accordance with wire 101, specifically LZ21-DFT-25% BaSO₄. As illustrated in FIG. 2, wire 1002 including a core of barium sulfate demonstrates a substantial enhancement in wire radiopacity compared to the monolithic wires 1004, 1006 and 1008.

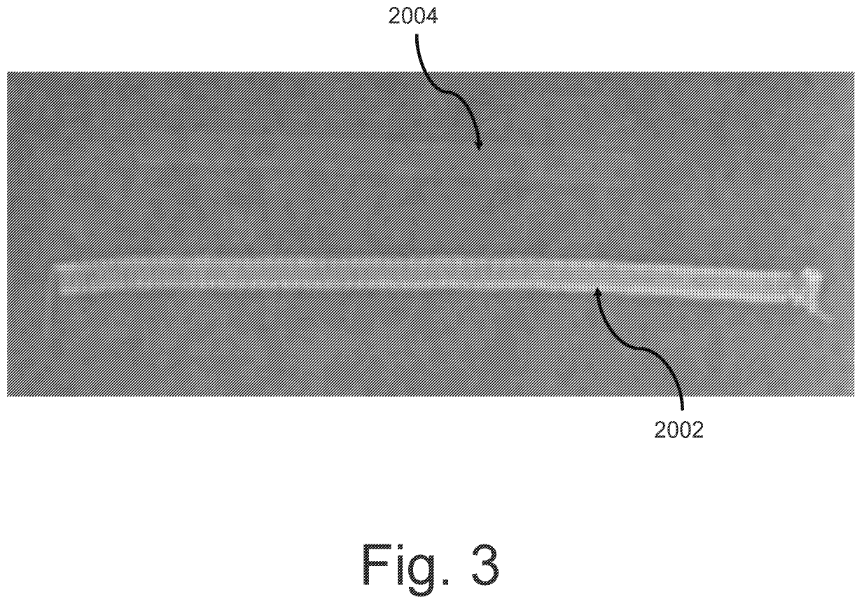

FIG. 3 illustrates 0.2 mm wire samples coiled into a helix having a 1.5 mm outer diameter. Wire 2004, shown at the top of FIG. 3, is made from monolithic LZ21, while wire 2002, shown at the bottom, is made from the LZ21-DFT-25% BaSO₄ composite wire in accordance with wire 101. As illustrated in FIG. 3, the coiled wire 2002 including a core of barium sulfate demonstrates a substantial enhancement in wire radiopacity compared to the monolithic coiled wire 2004.

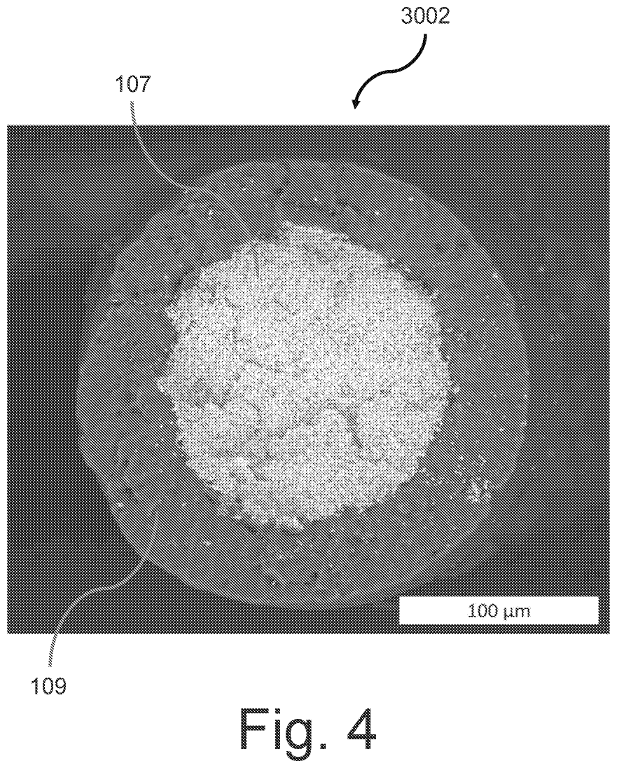

In another exemplary embodiment, Mg alloy ZX10 tubes are filled with a powder of either bismuth trioxide (Bi₂O₃) or tantalum oxide (Ta₂O₅) powders. These powder-filled tubes are drawn to diameters as small as 0.16 mm in accordance with the wire drawing principles discussed below. FIG. 4 is a photograph of a cross section of a resulting wire 3002, and in particular, illustrates a tensile fracture face of a wire 3002 having an overall diameter of 0.25 mm and including a ZX10 shell 109 and a Ta₂O₅ powder core 107. Wire 3002 is one embodiment of wire 101 discussed herein. As illustrated in FIG. 4, which is drawn to scale according to the scale in the lower right portion of the figure, the resulting core percentage (i.e., the percentage of the overall cross-section area occupied by core 107) is approximately 30% of the overall cross-sectional area of wire 3002, including both core 107 and shell 109.

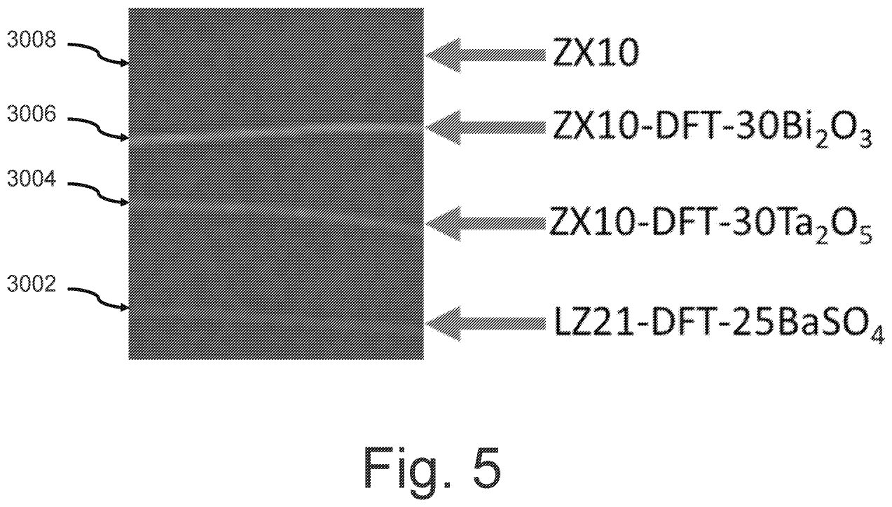

FIG. 5 illustrates X-ray imaging for three wires 3002, 3004, 3006 made in accordance with the present disclosure, and one predicate wire 3008. Wire 3002 is LZ21-DFT-25% BaSO₄, also discussed above with respect to FIG. 3. Wire 3004 is ZX10-DFT-30% Ta₂O₅, also discussed above with respect to FIG. 4. Wire 3006 is ZX10-DFT-30% Bi₂O₃, i.e., a wire in which shell 109 is ZX10 representing about 70% of the overall cross-section of wire 3006, and core 107 is Bi₂O₃ powder, representing about 30% of the overall cross-section of wire 3006. Wire 3008, included as a control sample, is a monolithic wire made of solid ZX10.

As shown in FIG. 5, a substantial enhancement in radiopacity is derived from the inclusion of Bi₂O₃, Ta₂O₅, and BaSO₄ powders as the cores 107 of wires 3002, 3004 and 3006, while the solid Mg alloy wire of wire 3008 is not visible in the X-ray image.

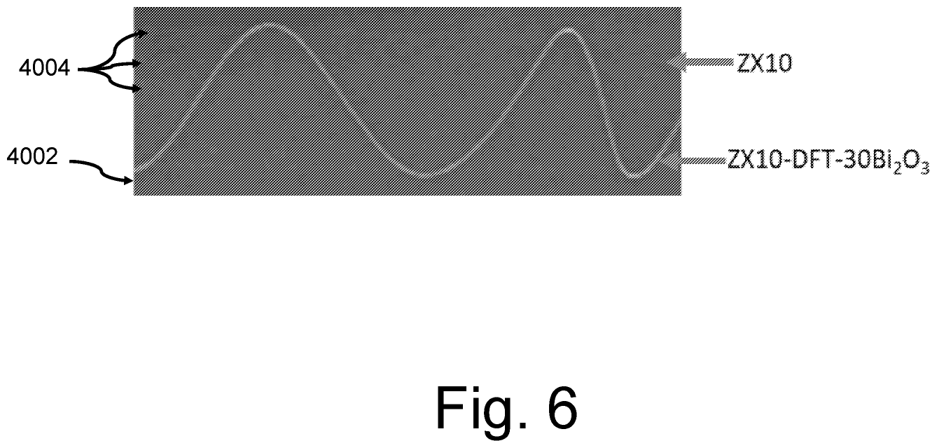

Turning to FIG. 6, an X-ray image of a 16-end braid of 0.16 mm diameter ZX10 wires 4004 is shown wrapped with a single ZX10-DFT-40Bi₂O₃ wire 4002. Consistent with the nomenclature herein, ZX10-DFT-40Bi₂O₃ wire 4002 has a shell 109 of ZX10 representing about 60% of the overall cross-section of wire 4002, while core 107 is Bi₂O₃ powder, representing about 40% of the overall cross-section of wire 4002. As shown in FIG. 6, a substantial enhancement in radiopacity is derived from the inclusion of Bi₂O₃ powder as the cores 107 of wire 4002, while the entire braid made from solid Mg alloy wires 4004 is only marginally visible in the X-ray image. FIG. 6 demonstrates the usefulness of wires 101 in the context of a radiopacity for a stent or other braided or woven construct, even where only some of the wires used for the multi-wire construct are made in accordance with the present disclosure.

As noted above, NiTi or other superelastic materials may also be used for shell 109 of wire 101. For example, wires 101 may use shell 109 formed of NiTiNb, a high-performance superelastic material suitable for use in vivo. Further discussion of NiTiNb material suitable for use with the present disclosure can be found in A Ni-Free β-Ti Alloy With Large and Stable Room Temperature Super-Elasticity by Cai et al.

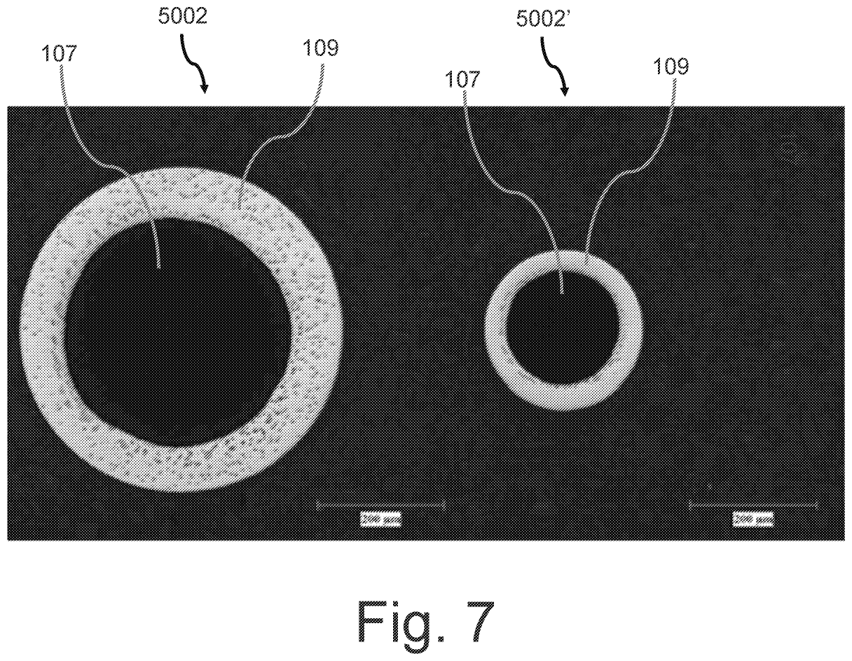

In one exemplary embodiment, a high strength NiTiNb alloy tube is filled with BaSO₄ powder and drawn to create a NiTiNb-DFT-60BaSO₄ wire 5002, i.e., 60% of the cross sectional area of the wire is occupied by the core 107 made of BaSO₄ powder. A pair of sample wires 5002 are shown in cross-section in FIG. 7, which illustrates a wire 5002 having an overall outer diameter of 0.5 mm as shown in the left image, and a wire 5002′ having an overall outer diameter of 0.25 mm as shown in the right image.

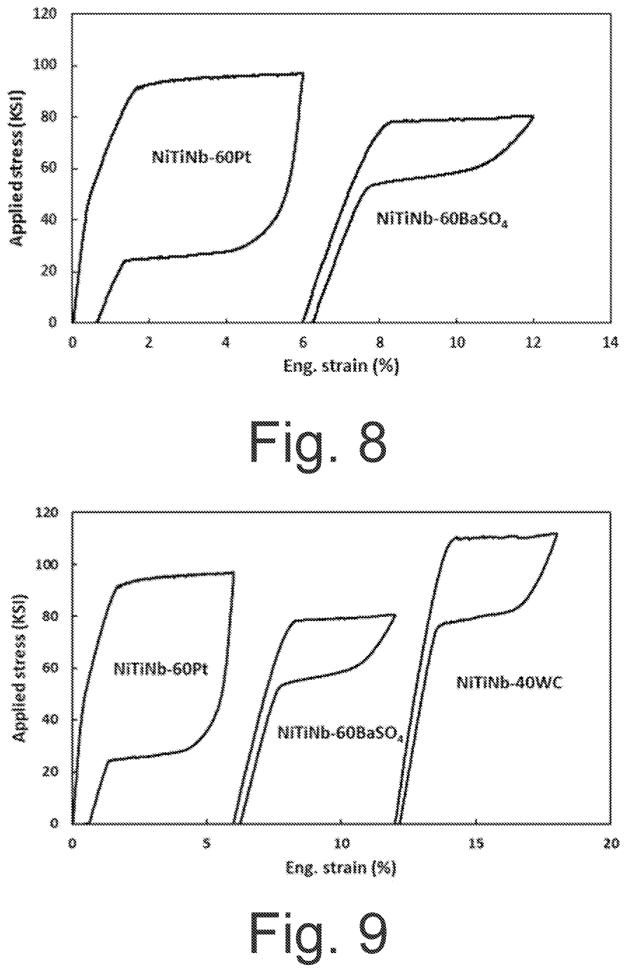

This drawn composite wire 5002 is then annealed at 400° C. for 5 minutes to impart superelastic properties to the NiTiNb shell 109. The composite wire is cyclically tensile tested to determine upper plateau stress, lower plateau stress, ultimate stress, and permanent set. This material was compared to a NiTiNb-DFT-60Pt core as reference. The resulting mechanical properties are illustrated at FIG. 8 with the graph from wire 5002 (tested at 0.25 mm diameter) shown at right and the reference material shown at left. As illustrated, from zero to six percent engineering strain, wire 5002 shows a significant increase in the lower plateau stress and a reduction in the permanent set as compared with the solid-core reference material. This results from the lack of mechanical resistance from the BaSO₄ core 107, as compared with the solid Pt core of the reference composite wire.

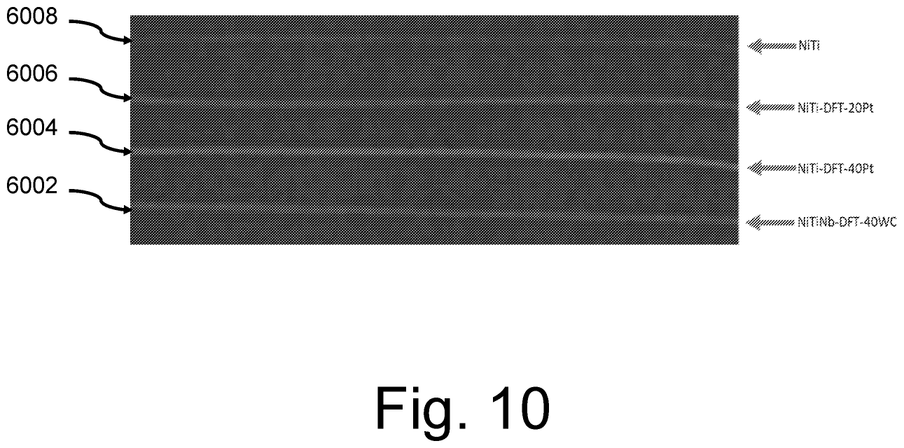

In yet another exemplary embodiment, a NiTiNb-DFT-40WC composite wire 6002 (FIG. 10) is produced similarly to wires described herein, i.e., 40% of the cross sectional area of the wire 6002 is occupied by the core 107 made of tungsten carbide powder. Samples of wire 6002 were produced in diameters of 0.25 mm for tensile testing and 0.1 mm for x-ray analysis.

Tensile test results, shown in FIG. 9, indicate that the tungsten carbide powder core 107 provided zero mechanical resistance to the NiTiNb shell 109. Additionally, FIG. 10 shows that the use of tungsten carbide powder for core 107 did enhance the radiopacity over standard nitinol, shown as reference wire 6008. Wire 6002 also demonstrated approximately the same radiopacity as NiTi-DFT-20Pt, shown as reference wire 6006, and slightly less radiopacity than NiTiNb-DFT-40Pt, shown as reference wire 6004. Wires 101 made in accordance with the present disclosure can therefore match the radiopacity of predicate composite wires having a solid-Pt core by enlarging the powder core 107, which can be done without mechanical penalties as described in detail above. Moreover, this lack of mechanical penalty can allow for similar radiopacity and enhanced deployment and mechanical performance over the service life of a medical device.

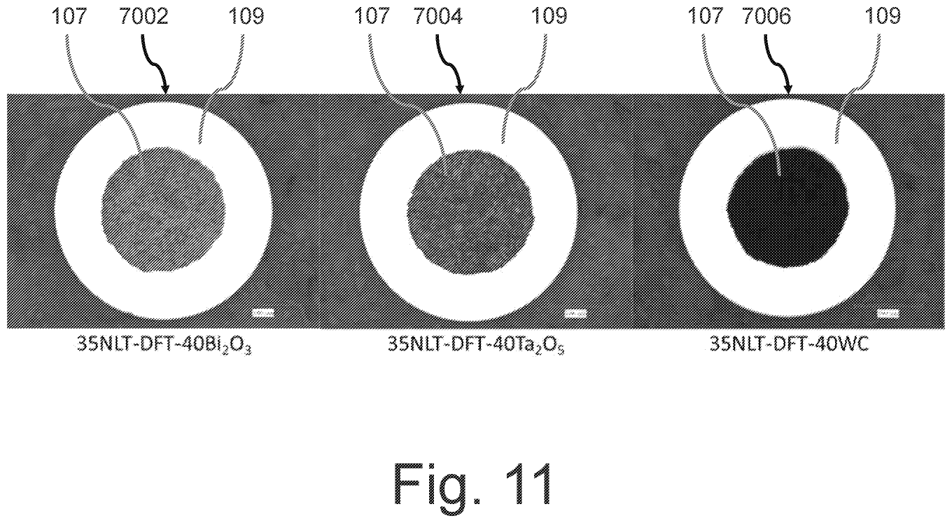

Wires 101 may use a shell 109 formed of Co—Ni—Cr—Mo alloys, such as 35N LT® referenced above. In an exemplary embodiment, 35N LT® tubes are filled with either Bi₂O₃, Ta₂O₅, or WC powders and drawn using drawing practices as described below to create 35NLT-DFT-40% Bi₂O₃, shown in FIG. 11 as wire 7002, 35NLT-DFT-40% Ta₂O₅, shown in FIG. 11 as wire 7004, or 35NLT-DFT-40% WC, shown in FIG. 11 as wire 7006. FIG. 11 shows cross-sectional cross sections of wire samples produced with diameters of 0.320 mm. The cores 107 nominally occupy 40% of the overall cross-sectional area of each of the wires 7002, 7004 and 7006, but can be measured at around 33% in the scale depictions of FIG. 11.

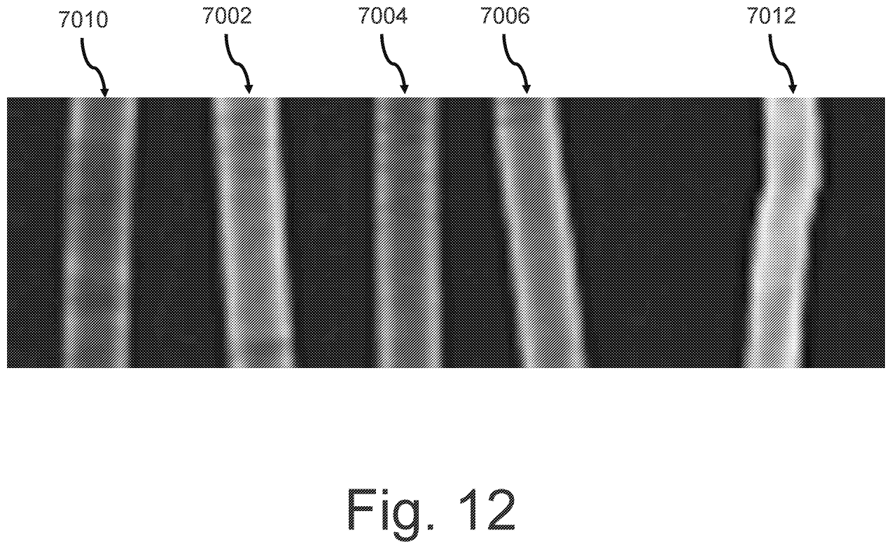

Wires 7002, 7004 and 7006 were produced with a 0.100 mm overall outer diameter for x-ray analysis, together with references wire 7010 made of solid 35N LT® and reference wire 7012 made of 35NLT-DFT-40Pt where the core is a solid, rather than powdered, material. For a first x-ray analysis, each of wires 7002, 7004, 7006, 7010, 7012 were coiled around 1 mm mandrels and imaged with an X-ray to generate the view of FIG. 12. As can be seen, wires 7002, 7004 and 7006 offer enhanced radiopacity as compared to wire 7010 made of solid 35N LT. Wires 7002 and 7006, having cores 107 made of powdered Bi₂O₃ and WC respectively, are more radiopaque than wire 7004 having core 107 made of powdered Ta₂O₅. The reference wire 7012 including a solid Pt core retained the highest radiopacity of the group, but with a mechanical penalty as described in detail above.

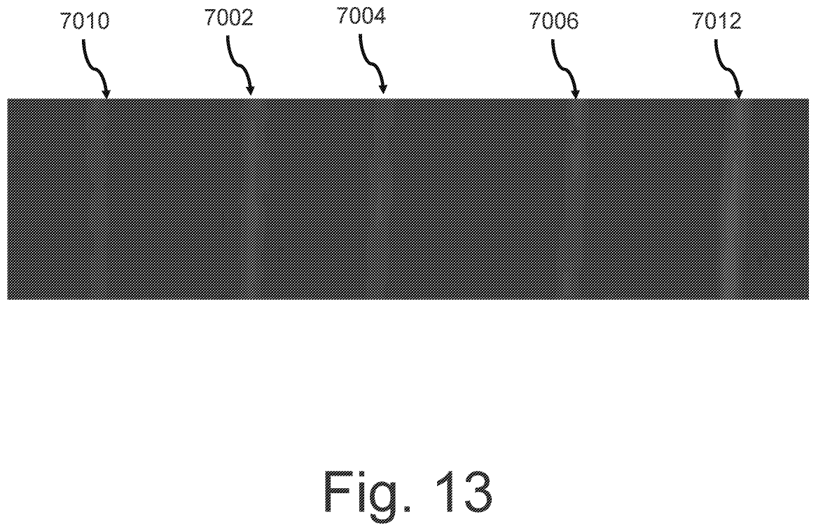

For a second X-ray analysis, the same set of wires 7002, 7004, 7006, 7010, 7012 in bare, straightened form and imaged with an X-ray to generate the view of FIG. 13. The same enhanced radiopacity of wires 7002, 7004 and 7006 can be seen as compared to wire 7010, with wires 7002 and 7006 being more radiopaque than wire 7004, and wire 7012 showing the highest radiopacity.

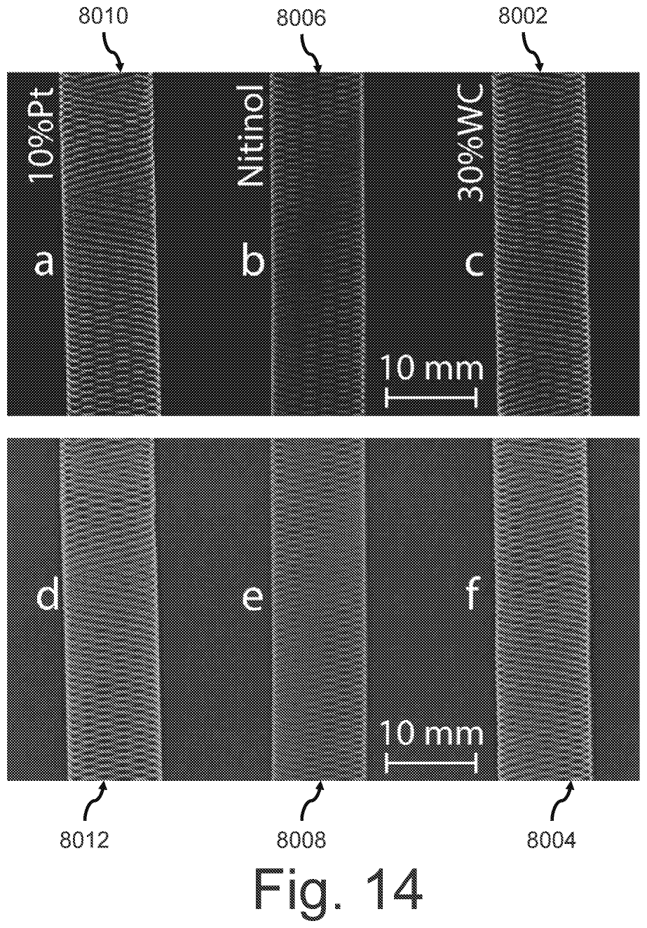

A further X-ray analysis was conducted for a series of 16-wire braids in which each wire of each braid is a round wire having a 0.18 mm diameter, generating the view of FIG. 14. Braid 8002 shown at part (c) of FIG. 14 includes NiTi-DFT-30% WC wires 101 made in accordance with the present disclosure, i.e., having a powder core 107 of tungsten carbide representing 30% of the overall cross-sectional area of the wire, and a NiTi shell 109 representing the remaining 70% of the cross-sectional area. Reference braids 8006 and 8010 included wires made of solid NiTi, shown at part (b) and NiTi-DFT-10% Pt (i.e., a 10% core made of solid platinum), shown at part (a). Braids 8002, 8006 and 8010 were subject to X-ray analysis at 60 kV for 20 mAs using a 12.5 mm aluminum body mimic. As illustrated, braid 8002 demonstrated comparable radiopacity to the Pt-core braid 8010, and superior radiopacity relative to the solid-wire NiTi braid 8006.

Braid 8004 shown at part (f) is made of the same material as braid 8002 described above. Braid 8008 shown at part (e) is made of the same material as braid 8006 described above. Braid 8012 shown at part (d) is made of the same material as braid 8010 described above. However, braids 8004, 8008 and 8012 were subjected to a different X-ray intensity, at 100 kV for 0.71 mAs using a 12.5 mm aluminum body mimic. As again illustrated, braid 8004 demonstrated comparable radiopacity to the Pt-core braid 8012, and superior radiopacity relative to the solid-wire NiTi braid 8008.

Relative biocompatibility of selected powders discussed herein was evaluated. In particular, ISO 10993-5 cytotoxicity testing was conducted via the agar overlay method on Bi₂O₃, Ta₂O₅, and WC powders. Bi₂O₃ and Ta₂O₅ showed no reactivity, and WC showed mild reactivity, with all receiving a “passing” assessment. As such, wires 101 made in accordance with the present disclosure, including the particular wires shown in the figures and discussed in detail above, are suitable for incorporation into in vivo medical devices.

### 3. Drawing and Cold Work

In an exemplary method of production, a hollow tube is procured in the desired materials for shell 109. Powder of a desired constituency is poured and tamped or otherwise compressed into the hollow tube such that the interior cavity of the tube is completely filled by the powder at a desired, evenly distributed density. This intermediate, or pre-form, material is then formed into a coarse wire structure by, for example, a schedule of drawing and annealing the intermediate material to create a structure ready for final processing into wire 101. Thereafter, the coarse wire structure may be subjected to one or more additional draws, as well as a final cold work conditioning step to form the finished and final construct, namely wire 101. One or more thermal processing steps such as shape setting, annealing and/or aging may then be performed in order to impart desired mechanical properties to the finished wire product, including superelasticity as discussed above. Further details of exemplary wire production and processing methods are further described below.

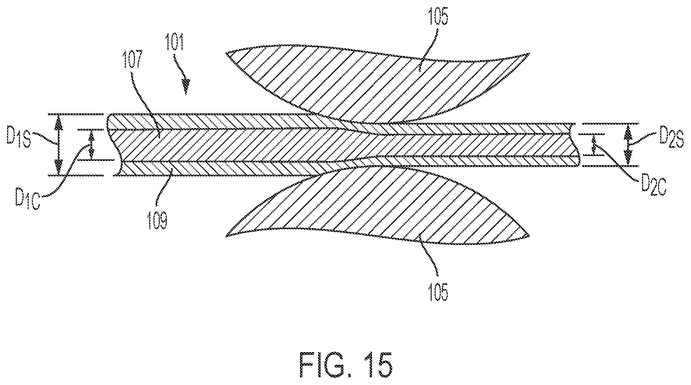

In one exemplary embodiment shown in FIG. 15, wire 101 made of medical-grade materials (described above) may be produced from a pre-form material into a wire of a desired diameter prior to final processing. That is, the pre-form material is drawn through one or more dies 105 (FIG. 15) to reduce the outer diameter of the intermediate material slightly while also elongating the material, after which the material is annealed to relieve the internal stresses (i.e., retained cold work as discussed below) imparted to the material by the drawing process. This annealed material is then drawn through one or more new dies 105 with a smaller finish diameter to further reduce the diameter of the material, and to further elongate the material. Further annealing and drawing of the material is iteratively repeated until the material is formed into a drawn wire construct ready for final processing into wire 101.

In an exemplary method of production, a tapered end is provided in the pre-form tubular structure made of the material selected for shell 109. This free end is placed protruding through the drawing die 105 and is then gripped and pulled through the die 105 to reduce the diameter of the construct and compresses the inner surface of shell 109 into the powder core 107. More particularly, the initial drawing process reduces the inner diameter of shell 109, such that shell 109 closes upon the outer diameter of core 107 and the inner diameter of shell 109 equals the outer diameter of core 107. After this initial drawing, the powder of the inner core 107 completely fills the central cavity of the outer shell 109 when viewed in section, as shown in FIG. 1. This drawing process is then iteratively repeated to further reduce the diameter of the material, which also further elongates the material. Iterative annealing and drawing of the material is performed until the material is formed into a drawn wire construct ready for final processing into a drawn composite wire 101. Further detail regarding the construction and geometry of a composite wire in accordance with the present disclosure can be found in U.S. Pat. Nos. 7,420,124, 7,501,579 and 7,745,732, filed Sep. 13, 2004, Aug. 15, 2005 and Jan. 29, 2009 respectively and all entitled DRAWN STRAND FILLED TUBING WIRE, the entire disclosures of which are hereby expressly incorporated herein by reference.

Drawn wire constructs are structurally distinguished from constructs formed by other methods (e.g., casting, machining, coating, etc.) by their characteristic smoothness and high reflectivity. In the case the composite wire 101 described herein (including additional particular wires, such as wires 3002, 4002, etc. described above), the circularity of the cross-section and the concentricity of the shell and core are substantially finer in a drawn construct as compared to, e.g., a coated construct. In addition, the microstructure of a drawn construct may be structurally distinct from other constructs, for example by exhibiting an elongated grain structure (shown in FIG. 17 and further discussed below) or a fine-grain structure after thermal processing.

The step of drawing subjects wire 101 to cold work. For purposes of the present disclosure, cold-working methods effect material deformation at or near room temperature, e.g. 20-30° C. In the case of composite wire 101, drawing imparts cold work to the material of both shell 109 and core 107, with concomitant reduction in the cross-sectional area of both materials. The total cold work imparted to wire 101 during a drawing step can be characterized by the following formula (I):

$$cw = 1 - {( \frac{{D}_{2}}{{D}_{1}} )}^{2} \times 100 \% \qquad ( I )$$

wherein “cw” is cold work defined by reduction of the original material area, “D_{2C}” and “D_{2S}” are the outer cross-sectional diameters of the core 107 and the shell 109 respectively after the draw or draws, and “D_{1C}” and “D_{1S}” are the outer cross-sectional diameters of the core 107 and the shell 109 respectively prior to the same draw or draws. Generally speaking, “D₂” as used in formula I means either D_{2C} or D_{2S}, depending on whether cold work cw is being computed for the core or shell respectively, and “D₁” as used in formula I means the corresponding D_{1C} or D_{1S}.

Referring to FIG. 15, the cold work step may be performed by the illustrated drawing process. As shown, wire 101 is drawn through a lubricated die 105 having an output diameter D_{2S}, which is less than diameter Dis of wire 101 prior to the drawing step. The outer diameter of wire 101 is accordingly reduced from pre-drawing diameter Dis to drawn diameter D_{2S}, thereby imparting cold work cw.

Alternatively, net cold work may be accumulated in wire 101 by other processes such as cold-swaging, rolling the wire (e.g., into a flat ribbon or into other shapes), extrusion, bending, flow forming, pilgering or cold-forging. Cold work may also be imparted by any combination of techniques including the techniques described here, for example, cold-swaging followed by drawing through a lubricated die finished by cold rolling into a ribbon or sheet form or other shaped wire forms. In one exemplary embodiment, the cold work step by which the diameter of wire 101 is reduced from D_{1S} to D_{2S} is performed in a single draw and, in another embodiment, the cold work step by which the diameter of wire 101 is reduced from D_{1S} to D_{2S} is performed in multiple draws which are performed sequentially without any annealing step therebetween.

For processes where the drawing process is repeated without an intervening anneal on composite wire 101, each subsequent drawing step further reduces the cross section of wire 101 proportionately, such that the ratio of the sectional area of shell 109 and core 107 to the overall sectional area of wire 101 is nominally preserved as the overall sectional area of wire 101 is reduced. Referring to FIG. 15, the ratio of pre-drawing core outer diameter D_{1C} to pre-drawings shell outer diameter Dis is the same as the corresponding ratio post-drawing. Stated another way, D_{1C}/D_{1S}=D_{2C}/D_{2S}.

Further details regarding wire drawing are discussed in U.S. patent application Ser. No. 12/395,090, filed Feb. 27, 2009, entitled “Alternating Core Composite Wire”, assigned to the assignee of the present invention, the entire disclosure of which is incorporated by reference herein. Drawing apparatuses and techniques are further described in International Patent Application No. WO 2019217350, filed May 7, 2019 and entitled APPARATUS AND METHOD FOR METAL-MEDIATED CATALYSIS, the entire disclosure of which is hereby incorporated herein by reference.

### 4. Annealing

Thermal stress relieving, otherwise known in the art as annealing, is achieved by heating the material to a nominal temperature not exceeding the melting point of the material or materials used in the construct. Annealing is used to improve the ductility of the construct between drawing steps, thereby allowing further plastic deformation by subsequent drawing steps. When calculating cold work cw using formula (I) above, it is assumed that no anneal has been performed subsequent to the process of imparting cold work to the material.

Heating wire 101 to a temperature sufficient to cause recrystallization of grains eliminates accumulated cold work in solid metallic materials such as shell 109. The cold work imparted by each iterative cold work process is relieved by fully annealing the material between draws, thereby enabling the next iterative cold working process for materials which might otherwise become brittle by repeated draws or other cold work processing. In full annealing, the cold-worked material is heated to a temperature sufficient to substantially fully relieve the internal stresses stored in the material, thereby relieving the stored cold work and “resetting” cold work to zero.

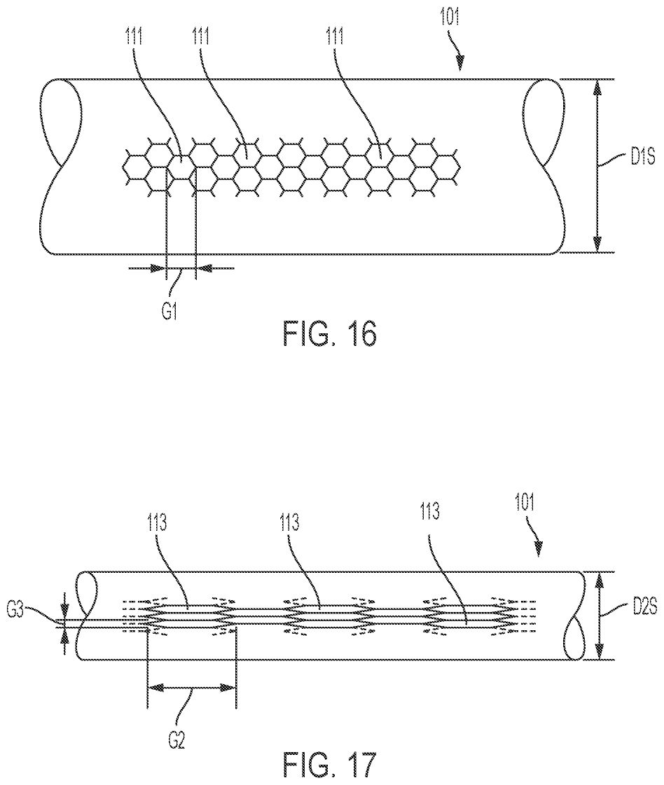

On the other hand, wire 101 subject to drawing or other mechanical processing without a subsequent annealing process retains an amount of cold work. The amount of retained cold work depends upon the overall reduction in diameter from Dis to Des, and may be quantified on the basis of individual grain deformation within the material as a result of the cold work imparted. Referring to FIG. 16, wire 103 is shown in a post-annealing state, with grains 111 shown substantially equiaxed, i.e., grains 111 define generally spheroid shapes in which a measurement of the overall length G1 of grain 111 is the same regardless of the direction of measurement. After drawing wire 101 (as described above), equiaxed grains 111 are converted into elongated grains 113 (FIG. 17), such that grains 113 become longitudinal structures defining an elongated grain length G2 (i.e., the longest dimension across grain 113) and a relatively shorter grain width G3 (i.e., the shortest dimension across grain 113). The elongation of grains 113 results from the cold working process, with the longitudinal axis of grains 113 generally aligned with the direction of drawing, as illustrated in FIG. 17.

The retained cold work of wire 101 after drawing can be expressed as the ratio of the elongated grain length G2 to the width G3, such that a larger ratio implies a grain which has been “stretched” farther and therefore implies a greater amount of retained cold work. By contrast, annealing wire 101 after an intermediate drawing process recrystallizes the material, converting elongated grains 113 back to equiaxed grains 111 and “resetting” the retained cold work ratio to 1:1 (i.e., no retained cold work).

For the above-described solid metals and solid metal alloys used for shell 109, full annealing or stress-relief annealing sufficient to tune strength and straightness properties may be accomplished at a temperature between and a time dependent on material and the geometry of the wire, such as the outer diameter D_{2S} of wire 101. Higher temperatures are associated with full annealing and lower temperatures associated with stress-relief annealing that does not fully recrystallize elongated grains 113 back to equiaxed grains 111. Annealing time, also called the “dwell time” during which the wire is exposed to the annealing temperature, is dependent on the size of the wire 101 and the desired effect of the annealing process, as well-understood by a person of skill in the art of material processing.

For purposes of the present discussion, annealing time may be assumed to be positively linearly correlated with the cross-sectional area of the wire being annealed. Thus, for a given annealing temperature, a similar annealing result is assumed for a first wire having a first cross-sectional area and annealed for a first amount of time, as for a second wire having twice the cross-sectional area of the first wire and annealed for a second amount of time that is twice the first time. However, for smaller fine wires and ultrafine wires, such as those having 200 μm or less, it may be assumed that the wire material becomes quickly heated through to the desired temperature, and the time for this heating is not significantly diameter-dependent. Thus, for wires 101 having diameters D_{2S} less than 200 μm, the annealing time is not correlated to diameters D_{2S} and is instead solely determined on the desired effect, i.e., full annealing or various gradations of stress-relief annealing as described above.

Moreover, annealing parameters can be expected to vary for varying wire diameters and wire configurations, with smaller diameters shortening the time of anneal for a given temperature and a given wire material. Whether a full anneal has been accomplished for any given wire sample can be verified in a number of ways as well known in the art, such as microstructural examinations using scanning electron microscopy (SEM), mechanical testing for ductility, strength, elasticity, etc., and other methods. Notably, the annealing parameters will affect the conditions of the powder core 107, but the core 107 will not experience cold work and grain deformation in the same way because the grains of the powder are free to rearrange around one another during cold work processing.

Further discussion of cold working and annealing methods can be found in U.S. Pat. No. 8,840,735, filed Sep. 18, 2009 and entitled FATIGUE DAMAGE RESISTANT WIRE AND METHOD OF PRODUCTION THEREOF, the entire disclosure of which is hereby incorporated by reference.

### 5. Applications

DFT-type, powder-cored composite wires in accordance with the present disclosure, including wire 101 and its various exemplary forms described above, may be used in a wide variety of smaller devices deployed via a transcatheter method. This could include neurovascular stents, flow diverters, and occluders, coronary stents, peripheral stents & stent grafts, valve frames, septal occluders, and venous filters. In the gastrointestinal system; devices for the upper and lower digestive tract could benefit. The enhanced radiopacity provided by wire 101 could also be useful in interventional devices such as guidewires, catheters, and stylets, to name a few.

The thin wall section for shell 109, enabled by the lack of mechanical interference from core 107 as described above, could also create a more uniform stress-strain distribution across wires used in torsion, providing improved stability and durability for mechanically intensive applications.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

### Aspects

Aspect 1 is a composite wire including a shell made of a solid metallic material, the shell defining an outer diameter and a hollow cavity extending longitudinally along the shell and defining an inner diameter; and a core made of a non-metallic powder material received within the hollow cavity and defining an outer diameter equal to the inner diameter.

Aspect 2 is the composite wire of Aspect 1, wherein the shell and the core are made of medical-grade materials.

Aspect 3 is the composite wire of any of Aspects 1 or 2, wherein the core is centered in the hollow cavity.

Aspect 4 is the composite wire of any preceding Aspect, wherein the core is a powder consisting of fine, dry grains having a substantially consistent density throughout a length of the hollow cavity of the shell.

Aspect 5 is the composite wire of any preceding Aspect, wherein the solid metallic material is one of magnesium, zinc, iron, titanium, titanium-beta, nitinol, stainless steel, cobalt-chrome, nickel, tantalum, platinum, tungsten, and alloys thereof.

Aspect 6 is the composite wire of any preceding Aspect, wherein the solid metallic material is bioabsorbable.

Aspect 7 is the composite wire of Aspect 6, wherein the solid metallic material is Mg or an Mg alloy.

Aspect 8 is the composite wire of any preceding Aspect, wherein the non-metallic powder material is one of bismuth trioxide, tantalum oxide, and tungsten carbide.

Aspect 9 is the composite wire of any Aspects 1-7, wherein the non-metallic powder material is one of barium sulfate, zirconium oxide, hafnium oxide, titanium oxide, niobium oxide, strontium oxide, zinc oxide, rare earth oxides, iodine or iodine-based compounds, bromine or bromine-based compounds.

Aspect 10 is the composite wire of any preceding Aspect, wherein a ductility, strength, fatigue endurance, plateau stress levels and/or permanent set of the composite wire is less than 5% different from a corresponding ductility, strength, fatigue endurance, plateau stress levels and/or permanent set of the shell alone.

Aspect 11 is the composite wire of any preceding Aspect, wherein the core defines at least 10% of an overall cross-sectional area of the shell and the core combined.

Aspect 12 is the composite wire of any preceding Aspect, wherein the non-metallic powder of the core has a higher radiopacity than the metallic material of the shell such that the wire has a radiopacity at least 100% greater than an equivalent solid wire made of the shell material only.

Aspect 13 is the composite wire of any preceding Aspect, wherein the solid metallic material of the shell and the non-metallic powder material of the core form a galvanic couple defining a voltage of less than 0.5V.

Aspect 14 is the composite wire of any preceding Aspect, wherein the composite wire is a drawn construct.

Aspect 15 is a drawn-filled tube wire construct, including a shell made of a shell material, the shell defining an outer diameter and a hollow cavity extending longitudinally along the shell and defining an inner diameter, and a core of made of a powder material received within the hollow cavity and defining an outer diameter equal to the inner diameter, wherein the non-metallic powder of the core has a higher radiopacity than the metallic material of the shell such that the wire has a radiopacity at least 100% greater than an equivalent solid wire made of the shell material only.

Aspect 16 is a drawn-filled tube wire construct, including a shell made of a shell material, the shell defining an outer diameter and a hollow cavity extending longitudinally along the shell and defining an inner diameter, and a core of made of a powder material received within the hollow cavity and defining an outer diameter equal to the inner diameter, the powder material forming a galvanic couple with the shell material defining a voltage of less than 0.5V.

Aspect 17 is the medical device comprising a wire in accordance with any preceding Aspect.

Aspect 18 is a wire for a medical device including a shell made of a shell material, the shell defining an outer diameter and a hollow cavity extending longitudinally along the shell and defining an inner diameter, the shell made of an absorbable metallic material, and a core received within the hollow cavity and defining an outer diameter equal to the inner diameter the core made of a non-metallic powder. The non-metallic powder of the core has a higher radiopacity than the metallic material of the shell such that the wire has a radiopacity at least 100% greater than an equivalent solid wire made of the shell material only. The core and the shell have a galvanic couple of less than 0.1V. The wire is configured to transition in vivo from a non-degraded configuration in which the shell is in solid form and encapsulates the non-metallic powder of the core, to a fully-degraded configuration in which the shell is absorbed and the non-metallic powder is exposed, the wire having a first mechanical strength in the non-degraded configuration and a substantially zero mechanical strength in the fully-degraded configuration.

Aspect 19 is a wire for a medical device including a shell made of a shell material, the shell defining an outer diameter and a hollow cavity extending longitudinally along the shell and defining an inner diameter, the shell made of a superelastic metallic material, and a core received within the hollow cavity and defining an outer diameter equal to the inner diameter the core made of a non-metallic powder. The non-metallic powder of the core has a higher radiopacity than the metallic material of the shell such that the wire has a radiopacity at least 100% greater than an equivalent solid wire made of the shell material only. The core and the shell having a galvanic couple of less than 0.1V. The wire defines a first isothermally recoverable strain which is substantially identical to a second isothermally recoverable strain of the equivalent solid wire made of the shell material only, whereby the core provides no mechanical resistance to a superelastic function of the shell such that the shell recovers from a mechanical deformation to the same extent as an equivalent diameter solid wire of the superelastic metallic material would recover from the mechanical deformation.

1. A composite wire comprising:
a shell made of a solid metallic material, the shell defining an outer diameter and a hollow cavity extending longitudinally along the shell and defining an inner diameter; and
a core made of a non-metallic powder material received within the hollow cavity and defining an outer diameter equal to the inner diameter.

2. The composite wire of claim 1, wherein the shell and the core are made of medical-grade materials.

3. The composite wire of claim 1, wherein the core is centered in the hollow cavity.

4. The composite wire of claim 1, wherein the core is a powder consisting of fine, dry grains having a substantially consistent density throughout a length of the hollow cavity of the shell.

5. The composite wire of claim 1, wherein the solid metallic material is one of magnesium, zinc, iron, titanium, titanium-beta, nitinol, stainless steel, cobalt-chrome, nickel, tantalum, platinum, tungsten, and alloys thereof.

6. The composite wire of claim 1, wherein the solid metallic material is bioabsorbable.

7. The composite wire of claim 6, wherein the solid metallic material is Mg or an Mg alloy.

8. The composite wire of claim 1, wherein the non-metallic powder material is one of bismuth trioxide, tantalum oxide, and tungsten carbide.

9. The composite wire of claim 1, wherein the non-metallic powder material is one of barium sulfate, zirconium oxide, hafnium oxide, titanium oxide, niobium oxide, strontium oxide, zinc oxide, rare earth oxides, iodine or iodine-based compounds, bromine or bromine-based compounds.

10. The composite wire of claim 1, wherein a ductility, strength, fatigue endurance, plateau stress levels and/or permanent set of the composite wire is less than 5% different from a corresponding ductility, strength, fatigue endurance, plateau stress levels and/or permanent set of the shell alone.

11. The composite wire of claim 1, wherein the core defines at least 10% of an overall cross-sectional area of the shell and the core combined.

12. The composite wire of claim 1, wherein the non-metallic powder of the core has a higher radiopacity than the metallic material of the shell such that the wire has a radiopacity at least 100% greater than an equivalent solid wire made of the shell material only.

13. The composite wire of claim 1, wherein the solid metallic material of the shell and the non-metallic powder material of the core form a galvanic couple defining a voltage of less than 0.5V.

14. The composite wire of claim 1, wherein the composite wire is a drawn construct.

15. A drawn-filled tube wire construct, comprising:
a shell made of a shell material, the shell defining an outer diameter and a hollow cavity extending longitudinally along the shell and defining an inner diameter; and
a core of made of a powder material received within the hollow cavity and defining an outer diameter equal to the inner diameter, wherein the non-metallic powder of the core has a higher radiopacity than the metallic material of the shell such that the wire has a radiopacity at least 100% greater than an equivalent solid wire made of the shell material only.

16. A drawn-filled tube wire construct, comprising:
a shell made of a shell material, the shell defining an outer diameter and a hollow cavity extending longitudinally along the shell and defining an inner diameter; and
a core of made of a powder material received within the hollow cavity and defining an outer diameter equal to the inner diameter, the powder material forming a galvanic couple with the shell material defining a voltage of less than 0.5V.

17. A medical device comprising a wire in accordance with claim 1.

18. A wire for a medical device comprising:
a shell made of a shell material, the shell defining an outer diameter and a hollow cavity extending longitudinally along the shell and defining an inner diameter, the shell made of an absorbable metallic material; and
a core received within the hollow cavity and defining an outer diameter equal to the inner diameter the core made of a non-metallic powder,
wherein the non-metallic powder of the core has a higher radiopacity than the metallic material of the shell such that the wire has a radiopacity at least 100% greater than an equivalent solid wire made of the shell material only,
wherein the core and the shell having a galvanic couple of less than 0.1V,
wherein the wire is configured to transition in vivo from a non-degraded configuration in which the shell is in solid form and encapsulates the non-metallic powder of the core, to a fully-degraded configuration in which the shell is absorbed and the non-metallic powder is exposed, the wire having a first mechanical strength in the non-degraded configuration and a substantially zero mechanical strength in the fully-degraded configuration.

19. A wire for a medical device comprising:
a shell made of a shell material, the shell defining an outer diameter and a hollow cavity extending longitudinally along the shell and defining an inner diameter, the shell made of a superelastic metallic material; and
a core received within the hollow cavity and defining an outer diameter equal to the inner diameter the core made of a non-metallic powder,
wherein the non-metallic powder of the core has a higher radiopacity than the metallic material of the shell such that the wire has a radiopacity at least 100% greater than an equivalent solid wire made of the shell material only,
wherein the core and the shell having a galvanic couple of less than 0.1V, and
wherein the wire defines a first isothermally recoverable strain which is substantially identical to a second isothermally recoverable strain of the equivalent solid wire made of the shell material only, whereby the core provides no mechanical resistance to a superelastic function of the shell such that the shell recovers from a mechanical deformation to the same extent as an equivalent diameter solid wire of the superelastic metallic material would recover from the mechanical deformation.
